(19) United States
(12) Patent Application Publication
(10) Pub. No.: US 2014/0179956 A1
(43) Pub. Date: Jun. 26, 2014

(54) METHOD FOR PREPARING CATECHOL

(71) Applicant: China Petrochemical Development Corporation, Taipei (Taiwan), Taipei City (TW)

(72) Inventors: I-Hui Lin, Taipei City (TW); Cheng-Fa Hsieh, Taipei City (TW); Pin-To Yao, Taipei City (TW)

(73) Assignee: China Petrochemical Development Corporation, Taipei (Taiwan), Taipei City (TW)

(21) Appl. No.: 14/102,937

(22) Filed: Dec. 11, 2013

(30) Foreign Application Priority Data
Dec. 25, 2012 (TW) 101149708

(51) Int. Cl. C07C 37/58

(52) U.S. Cl. CPC C07C 37/58
USPC 568/771

(57) ABSTRACT

A method for preparing catechol is provided. The method includes performing hydroxylation of phenol by using zirconium-containing titanium silicalite as a catalyst in the presence of phenol, a solvent and hydrogen peroxide. The method uses zirconium-containing titanium silicalite as a catalyst to increase the selectivity of phenol and utilization of hydrogen peroxide, and thus to increase the overall reaction yield.

### FIELD OF THE INVENTION

The present invention relates to methods for preparing catechol, and more particularly to a method for preparing hydroquinone and pyrocatechol by catalyzing phenol to undergo hydroxylation.

### BACKGROUND OF RELATED ART

Hydroquinone and pyrocatechol are important products in chemical industry, which can be applied in electronic, pharmaceutical or various chemical industries depending on the different properties thereof. Hydroquinone and pyrocatechol are also widely applied in the organic synthesis industry to prepare developing agents, polymerization blockers, skin whitening agents, antioxidants, bacteriocides, rubber auxiliaries, electroplating additives, light stabilizers, dyes, aromatic reducing agents, specific inks, and the like.

In the conventional methods for producing hydroquinone and pyrocatechol, hydroquinone and pyrocatechol are obtained by using hydrogen peroxide as an oxidant to hydroxylate phenol. During the reaction, a catalyst is added to enhance hydroxylation. Currently, in the hydroxylation of phenol, zeolite is often used as a catalyst since zeolite is easily separated from the product. The commonly used zeolite is, for example, titanium silicalite such as Ti-S-1, Ti-S-2 and Ti-β zeolite (molecular sieve), wherein the TS-1 (Ti-S-1) molecular sieve is commercialized.

UK Patent No. 2116974 discloses a method for hydroxylating phenol, in which 50 g of phenol, 39 g of acetone and 2.5 g of a TS-1 catalyst are mixed and added with 25 mL of 36% of hydrogen peroxide. Hydroxylation of phenol is performed at 80° C. At the end of the reaction, the conversion rate of phenol is 36.64%, the selectivity of catechol is 91.29%, and the yield of hydrogen peroxide is 68.9%. European Patent No. 0266825 discloses that 15.4 g of 60% of hydrogen peroxide is added dropwise spanning 45 minutes to a mixture of 99.8 g of phenol, 24.2 g of water, 18.5 g of acetone and 5.5 g of a gallium-containing TS-1 catalyst at 100 ° C., and the hydroxylation is performed for 60 minutes. At the end of the reaction, the yield of hydrogen peroxide is 74.4%. European Patent No. 0226257 discloses that 10.5 g of 35% of hydrogen peroxide is added dropwise spanning 45 minutes to a mixture of 56.7 g of phenol, 8.4 g of water, 13.5 g of acetone and 2.3 g of an aluminum-containing TS-1 catalyst at 100° C., and the hydroxylation is performed for 55 minutes. At the end of the reaction, the yield of catechol is 13.31%.

However, although the TS-1 solid catalysts used in the aforesaid patents are applied in the hydroxylation of catechol, there still exist the issues of low conversion rates of phenol and low selectivity of hydrogen peroxide. Accordingly, there is a need to provide a catalyst capable of increasing the conversion rate of phenol and the effective utilization of hydrogen peroxide in the hydroxylation of phenol.

### SUMMARY OF THE INVENTION

The present invention provides a method for preparing catechol, which includes performing hydroxylation of phenol by using zirconium-containing titanium silicalite as a catalyst in the presence of phenol, a solvent and hydrogen peroxide.

The zirconium-containing titanium silicalite of the present invention is formed by a hydrothermal reaction of a zirconium source, a silicon source, a titanium source, a template agent and water. As compared with a conventional TS-1 solid catalyst, the zirconium-containing titanium silicalite of the present invention, when being used as a reaction catalyst in the hydroxylation of phenol to prepare hydroquinone and pyrocatechol, increases the conversion rate of phenol and the utilization efficiency of hydrogen peroxide.

### DETAILED DESCRIPTION OF THE PREFERRED EMBODIMENTS

In the following, embodiments are provided to illustrate the detailed description of the present invention. Those skilled in the art can easily conceive the advantages and effects of the present invention, based on the disclosure of the specification. The present invention can also be practiced or applied by referring to the other different embodiments. Each of the details in the specification can also be modified or altered in various ways in view of different aspects and applications, without departing from the spirit of the disclosure of the present invention.

In one embodiment, the present invention provides a method for preparing a catechol, which includes performing hydroxylation of phenol by using zirconium-containing titanium silicalite as a catalyst in the presence of phenol, a solvent and hydrogen peroxide.

In an example for preparing zirconium-containing titanium silicalite, a hydrothermal reaction of a zirconium source, a silicon source, a titanium source, a template agent and water is performed at a temperature in a range of from 160 to 200° C. for 96 to 144 hours, to form a crystalline product. More specifically, the zirconium-containing titanium silicalite is formed by sequentially mixing and stirring a silicon source and a titanium source at a low temperature, adding a template solution to the mixture, adding an aqueous solution of a compound containing zirconium source, adding water while removing alcohol, adding silicon sol to conduct a hydrothermal reaction, separating the crystalline solid product from the liquid after the hydrothermal reaction is completed, rinsing the solid part with water until a neutral pH is reached, and baking and sintering the solid part.

In the preparation of the zirconium-containing titanium silicalite, the zirconium source can be zirconium salts or zirconium alkoxides. The silicon source can be silicate esters or polyethoxyl silane, and the titanium source can be tetraalkyl titanate.

For example, the zirconium source can be halide salts such as zirconium tetrafluoride, zirconium tetrachloride, zirconium tetrabromide and the like; acid salts such as zirconium carbonate, zirconyl nitrate, zirconium sulfate, zirconyl hydrochloride, zirconium phosphate and the like; an alkali such as zirconium hydroxide and the like; or zirconium alkoxides such as zirconium dipropoxide, zirconium tetra-n-butoxide, zirconium tetra-iso-butoxide and zirconium tetraethoxide.

For example, the silicate ester can be tetramethyl silicate, tetraethyl silicate, tetrapropyl silicate or tetrabutyl silicate. The polyethoxyl silane is, for example, ES-28 (n=1 or 2), ES-32 (n=3 or 4) or ES-40 (n=4 or 5).

In an exemplification of the titanium source, the tetraalkyl titanate is, for example, tetraethyl titanate, tetra-iso-propyl titanate and tetra-n-butyl titanate.

In the preparation of the catalyst of the present application, the template agent can be an aqueous solution or alcohol solution of tetra-n-propylammonium hydroxide or tetra-n-butylammonium hydroxide. Moreover, the template agent can be obtained by dissolving tetra-n-propylammonium bromide or tetra-n-butylammonium bromide in an aqueous solution or an alcohol solution, and then by using anionic exchange resins, wherein the alcohol can be one of the alcohols containing 1 to 5 carbon atoms or a mixture thereof. Generally, the concentration of tetra-n-propylammonium hydroxide or tetra-n-butylammonium hydroxide in the aqueous solution or alcohol solution can be in a range of from 5 wt % to 50 wt %, and preferably in a range of from 20 wt % to 40 wt %.

In an embodiment, in the catalyst of the present invention, a molar ratio of zirconium to silicon is in a range of from 0.0001 to 0.01, and a molar ratio of titanium to silicon is in a range of from 0.01 to 0.05.

In an embodiment of the present invention, the amount of zirconium-containing titanium silicalite used as a catalyst is in a range from 0.5 to 10 wt %, preferably from 1 to 8 wt %, and more preferably from 1.5 to 6.5 wt %, based on the total weight of phenol, the solvent and hydrogen peroxide.

Furthermore, the solvent used for the hydroxylation of phenol can be at least one selected from the group consisting of alcohols, ketones, nitriles, organic acids and water. Preferably, the solvent is water.

A molar ratio of hydrogen peroxide to phenol used in the hydroxylation of phenol is less than or equal to 1. In an embodiment, a molar ratio of hydrogen peroxide to phenol is in a range of from 0.1 to 1, and preferably in a range from 0.1 to 0.8, and more preferably in a range of from 0.25 to 0.65.

Generally, the hydroxylation of phenol is performed at a temperature in a range from 293K to 373K, and preferably in a range from 303K to 363K, or in a range from 328K to 348K.

In the example of the present invention for preparing catechol, the conversion rate of phenol achieves up to 48.5%, the selectivity of catechol achieves up to 96.65%, the selectivity of hydrogen peroxide achieves up to 92.65%, and the conversion rate of hydrogen peroxide is almost 100%. Accordingly, in the presence of the zirconium-containing titanium silicalite of the present invention, the hydroxylation of phenol to produce catechol achieves excellent reactivity.

The following examples are used to further illustrate the features and effects of the present invention, but they should not be construed to limit the scope of the present invention.

### EXAMPLES

The conversion rates, selectivity and yields disclosed in the specification of the present invention are calculated by the following equations:

Xₚₕ=conversion rate of phenol=molar number of consumed phenol/molar number of fed phenol×100%;

S_{dph}=selectivity of catechol=(molar number of produced hydroquinone+molar number of produced pyrocatechol)/molar number of consumed phenol×100%;

S_{BQ}=molar number of produced benzoquinone/molar number of consumed phenol×100%;

X_{H2O2}=conversion rate of hydrogen peroxide=molar number of consumed hydrogen peroxide/molar number of fed hydrogen peroxide ×100%; and

S_{H2O2}=selectivity of hydrogen peroxide=molar number of produced catechol/molar number of consumed hydrogen peroxide×100%.

### Preparation Example 1

### Synthesis of Zirconium-containing Titanium Silicalite A

A 250 mL round-bottomed beaker was sealed with nitrogen in a vacuum system, and the temperature of the round-bottomed beaker was cooled to 5° C. After the temperature reached a balance, 30.0 g of tetraethyl silicate, 56.00 g of tetra-n-propylammonium hydroxide (20 wt %), and 2.92 g of tetra-n-butyl titanate were placed in the round-bottomed beaker, and stirred for 1 hour to form a mixture. After stirring, 0.3225 g of zirconium sulfate tetrahydrate was dissolved in 44.0 g of water as a zirconium source. The solution of zirconium source was added dropwise to the mixture, stirred for 1 hour, and then stirred for an additional 1 hour at room temperature. Finally, alcohol was removed at 80° C. for 2 hours. 10.80 g of AS-40 silicon sol gel solution (40 wt % SiO₂) was dispersed in 73.0 g of water to form a dispersion. The synthetic gel upon alcohol removal in the round-bottomed beaker and the dispersion were mixed and stirred for 1 hour to obtain a mixed solution of zirconium-titanium-silicon-template synthetic gel. The mixed solution was sealed in a pressure-resistant stainless-steel tank lined with Teflon, and subjected to a hydrothermal treatment at 180° C. for 120 hours. The solid and liquid were separated, and the solid part was rinsed with water until a neutral pH was reached. The solid part was dried at 100° C., and sintered at 550° C. for 8 hours, to obtain the zirconium-containing titanium silicalite A.

### Preparation Example 2

### Synthesis of Zirconium-containing Titanium Silicalite B

Zirconium-containing titanium silicalite B was prepared in the same way as in preparation example 1, except that the amount of tetra-n-butyl titanate used was 1.46 g.

### Preparation Example 3

### Synthesis of Zirconium-containing Titanium Silicalite C

Zirconium-containing titanium silicalite C was prepared in the same way as in preparation example 1, except that the amount of tetra-n-butyl titanate used was 1.46 g, and the amount of zirconium sulfate tetrahydrate used was 0.0806 g.

### Comparative Example 1

### Synthesis of Titanium Silicalite D

250 mL round-bottomed beaker was sealed with nitrogen in a vacuum system, and the temperature of the round-bottomed beaker was cooled to 5° C. After the temperature reached a balance, 30.0 g of tetraethyl silicate, 56.00 g of tetra-n-propyl ammonium hydroxide (20 wt %) and 2.92 g of tetra-n-butyl titanate were placed in the round-bottomed beaker, and stirred for 1 hour. Then, 44.0 g of water was added dropwise into the round-bottomed beaker, the mixture was stirred for 1 hour, and the mixture was stirred at room temperature for an additional 1 hour. Finally, alcohol was removed from the mixture at 80° C. for 2 hours to form a synthetic gel. 10.84 g of AS-40 silicon sol gel solution (40 wt % SiO₂) was dispersed in 73.0 g of water to form a dispersion. The synthetic gel in the round-bottomed beaker and the dispersion were mixed and stirred for 1 hour, to obtain a mixed solution of titanium-silicon-template synthetic gel. The mixed solution was sealed in a pressure-resistant stainless-steel tank lined with Teflon, and subjected to a hydrothermal treatment at 180° C. for 120 hours. The solid and liquid were separated, and the solid part was rinsed with water until a neutral pH was reached. The solid part was dried at 100° C., and sintered at 550° C. for 8 hours, to obtain titanium silicalite D.

### Comparative Example 2

### Synthesis of Titanium Silicalite E

Titanium silicalite E was synthesized in the same way as in comparative example 1, except that the amount of tetra-n-butyl titanate used was 1.46 g.

### Example 1

The zeolites obtained in comparative examples 1 and 2 and preparation examples 1 to 3 were used as catalysts in the hydroxylation of phenol as follows. 0.178 mole of phenol, 1.066 mole of pure water and 1.844 g of a zeolite catalyst were placed in a 250 mL three-necked flask under nitrogen gas and the temperature was held at 333K. 0.089 mole of an aqueous solution of 35 wt % of hydrogen peroxide was fed by pumping for three hours, the reaction was performed for 3 hours, the temperature was lowered to room temperature after the reaction, the reaction fluid was separated from the zeolite catalyst, and the reaction composition was analyzed by using gas chromatography. The results are shown in Table 1.

TABLE 1

| Zeolite | | | | | |
|---|---|---|---|---|---|
| catalyst | Xₚₕ | S_{diph} | S_{BQ} | X_{H2O2} | S_{H2O2} |
| A | 46.80 | 88.38 | 0.62 | 100.00 | 83.14 |
| (Preparation | | | | | |
| Example 1) | | | | | |
| B | 45.06 | 96.65 | 0.88 | 100.00 | 87.75 |
| (Preparation | | | | | |
| Example 2) | | | | | |
| C | 48.04 | 93.55 | 2.29 | 100.00 | 89.88 |
| (Preparation | | | | | |
| Example 3) | | | | | |
| D | 44.07 | 89.01 | 4.18 | 99.61 | 78.13 |
| (Comparative | | | | | |
| example 1) | | | | | |
| E | 43.47 | 86.62 | 0.13 | 100.00 | 75.51 |
| (Comparative | | | | | |
| example 2) | | | | | |

### Example 2

The zeolites obtained in comparative example 2 and preparation example 3 were used as catalysts in the hydroxylation of phenol as follows. 0.178 mole of phenol, 1.066 mole of pure water and 1.844 mole of a zeolite catalyst were placed in a 250 mL three-necked flask under nitrogen gas and the temperature was held at 328K or 348K. 0.089 mole of an aqueous solution of 35 wt % of hydrogen peroxide was fed by pumping for three hours, the reaction was performed for 3 hours, the temperature was lowered to room temperature after the reaction, the reaction fluid was separated from the zeolite catalyst, and the reaction composition was analyzed by using gas chromatography. The results are shown in Table 2.

TABLE 2

| | Reaction | | | | | |
|---|---|---|---|---|---|---|
| Zeolite | temperature | | | | | |
| catalyst | (K) | Xₚₕ | S_{diph} | S_{BQ} | X_{H2O2} | S_{H2O2} |
| C | 328 | 48.50 | 95.77 | 1.95 | 100.0 | 92.65 |
| (Preparation | | | | | | |
| Example 2) | | | | | | |
| E | 328 | 32.37 | 89.39 | 5.12 | 100.0 | 57.98 |
| (Comparative | | | | | | |
| Example 2) | | | | | | |
| C | 348 | 46.34 | 89.64 | 0.20 | 100.0 | 83.01 |
| (Preparation | | | | | | |
| Example 3) | | | | | | |
| E | 348 | 43.31 | 84.24 | 0.20 | 100.0 | 78.20 |
| (Comparative | | | | | | |
| example 2) | | | | | | |

It is clear from the above results that when the zirconium-containing titanium silicalite catalyst of the present invention is used as a catalyst for the hydroxylation of phenol to produce catechol, the conversion rate of phenol and selectivity of hydrogen peroxide are increased.

The above examples are only used to illustrate the principle of the present invention and the effect thereof, and should not be construed as to limit the present invention. The above examples can be modified and altered by those skilled in the art, without departing from the spirit and scope of the present invention as defined in the following appended claims.

1. A method for preparing catechol, comprising:
performing hydroxylation of phenol by using zirconium-containing titanium silicalite as a catalyst in the presence of the phenol, a solvent and hydrogen peroxide.

2. The method of claim 1, wherein the catalyst is formed by a hydrothermal reaction of a zirconium source, a silicon source, a titanium source, a template agent and water.

3. The method of claim 2, wherein the hydrothermal reaction is performed at a temperature in a range of from 160 to 200° C. for 96 to 144 hours.

4. The method of claim 2, wherein a molar ratio of zirconium to silicon in the catalyst is in a range of from 0.0001 to 0.01, and a molar ratio of titanium to silicon in the catalyst is in a range of from 0.01 to 0.05.

5. The method of claim 2, wherein the zirconium source is a zirconium salt or a zirconium alkoxide, the silicon source is a silicate ester or polyethoxyl silane, and the titanium source is tetraalkyl titanate.

6. The method of claim 2, wherein the template agent is an aqueous solution or an alcohol solution of tetra-n-propylammonium hydroxide or tetra-n-butylammonium hydroxide.

7. The method of claim 6, wherein the tetra-n-propylammonium hydroxide or the tetra-n-butyl ammonium hydroxide is at a concentration in a range of from 5 to 50 wt% in the aqueous solution or the alcohol solution.

8. The method of claim 1, wherein an amount of the catalyst is in a range of from 0.5 to 10 wt % based on a total weight of the phenol, the solvent and the hydrogen peroxide.

9. The method of claim 1, wherein the hydroxylation is performed at a temperature in a range of from 293K to 373K.

10. The method of claim 1, wherein a molar ratio of the hydrogen peroxide to the phenol is in a range of from 0.1 to 1.

11. The method of claim 1, wherein the solvent is at least one selected from the group consisting of alcohols, ketones, nitriles, organic acids and water.
